(19) United States
(12) Patent Application Publication
(10) Pub. No.: US 2025/0381173 A1
(43) Pub. Date: Dec. 18, 2025

(54) Long-Lasting Anaesthetic Formulation

(71) Applicant: BERTIE INTERNATIONAL AB, Saltsjö-Boo (SE)

(72) Inventors: Christer ROLF, Saltsjö-Boo (SE); Ibrahim TURAN, Stockholm (SE)

(21) Appl. No.: 18/876,020

(22) PCT Filed: Jun. 16, 2023
(86) PCT No.: PCT/EP2023/066296

§ 371 Date: Dec. 17, 2024

(30) Foreign Application Priority Data
Jun. 17, 2022 (EP) 22179718.6

(51) Int. Cl. A61K 31/445; A61K 9/00; A61K 9/51; A61K 47/10; A61K 47/18; A61P 23/02

(52) U.S. Cl. CPC A61K 31/445; A61K 9/0019; A61K 9/51; A61K 47/10; A61K 47/18; A61P 23/02

(57) ABSTRACT

The present invention is within the technical field of pain-relief and relates to a pharmaceutical composition comprising at least one anesthetic agent selected from the group consisting of ropivacaine, bupivacaine, etidocaine, levobupivacaine, lidocaine, lignocaine, mepivacaine, articaine, dibucaine, levobupivacaine, prilocaine, benzocaine, chloroprocaine, cocaine, procaine, proparacaine, tetracaine and any pharmaceutically acceptable salt, hydrate, solvate or prodrug thereof; at least one alkanolamine selected from the group consisting of triethanolamine, tripropanolamine and trimethanolamine; water; and optionally a pharmaceutically acceptable diluent, carrier and/or excipient. The present disclosure furthermore relates to the use of the composition for providing pain-relief, a method of treatment, a method of producing said pharmaceutical composition as well as a carbon quantum dot formed from components of the pharmaceutical composition.

### TECHNICAL FIELD

The inventive concept described herein generally relates to pharmaceutical compositions comprising an anesthetic agent. Specifically, the present inventive concept relates to a pharmaceutical composition with a long-lasting anesthetic effect, and the production of such composition.

### BACKGROUND

Local anesthetic agents create an absence of pain in a specific location of the body without a loss of consciousness by reversibly inhibiting nerve impulses, causing a sensory or motor blockade. They are used to numb areas of the body when surgeries or painful examination by a physician are to be performed, or to provide pain-relief when needed. The agents may be divided into groups based on type. The ester group of local anesthetic drugs comprises benzocaine, chloroprocaine, cocaine, procaine, proparacaine, tetracaine. The amide group of local anesthetic drugs comprises articaine, bupivacaine, cinchocaine, etidocaine, levobupivacaine, lidocaine, mepivacaine, prilocaine, ropivacaine and trimecaine. The drugs have the similar mechanism of action in which they cause a reversible inhibition of sodium ion influx, which blocks impulse conduction in nerve fibers.

The drugs ropivacaine and bupivacaine are highly similar, with only one methyl group difference. However, ropivacaine benefits from higher efficacy, which may partly be because ropivacaine is sold as an optionally pure S-enantiomer.

Ropivacaine is a well-tolerated anesthetic drug, effective for surgical anesthesia as well as for relief of postoperative and labor pain (Kuthiala and Chaudhary, 2011). Carbon quantum dots are a class of nanomaterials that have attracted attention because of their low toxicity, water solubility and fluorescence. As such, it has been suggested that they may prove useful in the field of imaging and drug delivery. Qu et al describe the synthesis of ibuprofen-based carbon quantum dots and show that the dots are stable, non-toxic and have good bio-combability in water. Moreover, the anti-inflammatory properties of ibuprofen were retained.

Although local anesthetic agents are effective for surgical anesthesia and relief of pain, there is room for improvement, for example pharmacokinetic (PK) or pharmacodynamic (PD) properties in the body of a mammal. Although effort has been made in the field of anesthetic products, there is still a need for improved products. Would improved products be provided, an even greater benefit for mankind would arise if such products would be long-lasting.

### SUMMARY OF THE INVENTION

It is an object of the present invention to at least partly reduce or overcome challenges in the prior art, and provide means for treatment, alleviation and/or prevention of pain. These and other objects are achieved in full, or at least in part, by aspects of the inventive concepts as disclosed herein. The present inventor have surprisingly found that a pharmaceutical composition comprising at least one anesthetic agent selected from the group consisting of ropivacaine, bupivacaine, etidocaine, levobupivacaine, lidocaine, lignocaine, mepivacaine, articaine, dibucaine, levobupivacaine, prilocaine, benzocaine, chloroprocaine, cocaine, procaine, proparacaine, tetracaine and any pharmaceutically acceptable salt, hydrate, solvate or prodrug thereof; at least one alkanolamine selected from the group consisting of triethanolamine, tripropanolamine and trimethanolamine; water; and optionally a pharmaceutically acceptable diluent, carrier and/or excipient is efficient in providing a long-lasting anesthetic effect. Thus, in a first aspect of the present invention, there is provided such pharmaceutical composition, which benefits from an increase in duration of pain relief, when compared to a similar compound that is not formulated in accordance with the inventive composition. The inventor shows that a composition according to the present invention provides increased duration of pain-relief in vivo. It is envisioned that the increased pain-relief in vivo at least partly replaces a need for opioid treatments, which is commonly used in clinical practice as postoperative rescue medication. As such, risks of complications from opioid treatment may be decreased by use of a composition according to the present invention.

In one embodiment, said at least one anesthetic agent is selected from the group consisting of ropivacaine, bupivacaine, etidocaine, levobupivacaine, lidocaine, lignocaine, mepivacaine, articaine, dibucaine, levobupivacaine, prilocaine and any pharmaceutically acceptable salt, hydrate, solvate or prodrug thereof. In one embodiment, said at least one anesthetic agent is selected from the group consisting of ropivacaine, bupivacaine, etidocaine, levobupivacaine, lidocaine, lignocaine, mepivacaine and any pharmaceutically acceptable salt, hydrate, solvate or prodrug thereof. In one embodiment, said pharmaceutical composition further comprises polyethylene glycol (PEG).

Preferably, said composition comprises carbon quantum dots. Without being bound by theory, it is envisioned that the carbon quantum dots provide an increase of the therapeutic effect or provide improved pharmacokinetic and/or pharmacodynamic properties of an anesthetic agent in the inventive composition. Without being bound by theory, such an increase may be the result of increased water solubility; or increased cellular internalization of the anesthetic agent; or that said anesthetic agent is associated with said carbon quantum dot for a period of time, thus providing a slow-release effect of said anesthetic agent. In one embodiment, said carbon quantum dots comprises said at least one anesthetic agent. In one embodiment, said carbon quantum dots comprise at least one of said alkanolamine selected from the group consisting of triethanolamine, tripropanolamine and trimethanolamine. In one embodiment, said carbon quantum dots comprise triethanolamine.

In one embodiment, said carbon quantum dots further comprises PEG. In one embodiment, said PEG is located on the surface of said carbon quantum dots.

Several routes of administration are envisioned. In one embodiment, said composition is formulated to be administrated by injection or by topical administration.

In one embodiment, said composition is formulated to be administrated as a nerve block in lower and upper extremities, intraarticular in various joints, subcutaneous, intrathecal. Preferably, administration is not intravenous administration.

In one embodiment, said administration is by topical administration, such as by a patch, a cream, a gel or by a spray. Such administration may be beneficial since use of a topical composition may not require a medicinal practitioner to apply said pharmaceutical composition. As such, a subject in need of anesthesia may self-administer the composition to herself or himself. Self-administration has the advantage of allowing a patient to adjust the dose or the frequency of medication either according to a subjective evaluation of their condition or according to a schedule prescribed by a treating physician. The term “schedule prescribed by the treating physician” includes the alternative where a patient makes a subjective evaluation of his/her condition, either unaided or aided by a questionnaire or a range or scale, or using an algorithm or a computer program, indicating the suitable next dose.

Percutaneous administration, using the composition according to the present invention formulated as a cream, a gel, and an ointment or in the form of adhesive medicine patches, is another possible form of administration, similarly suitable for self-administration. The advantages of self-administration listed above apply also to percutaneous administration, with the added advantage that the administration can easily be interrupted if desired or necessary, e.g. by removing the medicine patch.

When the topical composition is in the form of a patch, a gel or a spray said composition may comprise additional ingredients. The skilled person understands what additional ingredients are suitable for the specific topical use. For example, a spray may need the addition of a propellant. Suitable propellants are apparent to the skilled person. A lotion, a skin cream or an ointment may need the addition of an emollient. Suitable emollients are apparent to the skilled person.

A gel may need the addition of a gelifying agent such as a gelifying polymer. Suitable polymers are apparent to the skilled person. For example, cellulose, such as a nonionic water-soluble cellulose ethers. In addition, a gel may comprise an alcohol, such as a C1-C4 alcohol, such as a C1-C3 alcohol or a C2-C4 alcohol, such as ethanol.

In addition, a gel may comprise benzyl alcohol. In some embodiments, the gel comprises Ropivacaine and Lidocaine. In some embodiments, the gel comprises ropivacaine and lidocaine, benzyl alcohol, ethanol and Klucel MFX. In addition, the gel may comprise Sodium Borate (Borax). An example of a gel comprising ropivacaine and lidocaine is described in Example 5.

A patch may comprise an additional agent and may comprise more than one local anesthetic agent. In one embodiment, when the topical composition is in the form of a patch, the composition comprises at least one of bupivacaine and ropivacaine, and furthermore lidocaine, such as that the composition comprises ropivacaine and lidocaine.

It is also possible that the composition is administered via a depot formulation, which releases an effective amount of the anesthetic agent as disclosed herein, over a period of time. The skilled person will appreciate that the depot formulation may be adapted to deliver the desired effective dose as prescribed by a treating physician. A depot formulation may be a subcutaneous depot formulation. Thus, in one embodiment, said administration is via a depot formulation, such as a subcutaneous depot formulation.

In one embodiment, said administration is by injection, such as by parenteral injection or by subcutaneous injection. In one embodiment, said administration is as a single injection. Because of the long lasting effect of the inventive composition, a single injection may be suitable when administering the composition of the invention, which may be time-efficient.

Conventionally used excipients for intravenous administration are for example sterile water for injections (WFI), sterile buffers (for example buffering the solution to pH 7.4), an albumin solution, lipid solutions, cyclodextrin and variants thereof, and the like. Conventionally used excipients for subcutaneous administration are for example sterile water for injections (WFI), sterile buffers (for example buffering the solution to pH 7.4), lipid solutions, cyclodextrins and the like. Conventionally used excipients for subcutaneous administration via a subcutaneous delivery system, such as a subcutaneous rod, are for example sterile water for injections (WFI), sterile buffers (for example buffering the solution to pH 7.4), lipid solutions, cyclodextrins and the like.

Conventionally used excipients for transdermal and/or subcutaneous administration are for example vaseline, liquid paraffin, glycerol, water, MCT oil, sesame oil and the like.

The skilled person will appreciate that the suitable dose will naturally vary depending on the mode of administration, the particular condition to be treated or the effect desired, gender, age, weight and health of the patient, as well as possibly other factors, evaluated by the treating physician. In one embodiment, the dose of said anesthetic agent is a single dose (a unit dose) of from 5 to 600 mg, such as from 10 to 500 mg, such as from 20 to 450 mg, such as from 50 to 400 mg, such as from 100 to 400 mg, such as from 200 to 350 mg, such as from 200 to 300 mg.

In one embodiment, the concentration of said anesthetic agent is at a dose of 1-20 mg/ml, such at a dose of 2-15 mg/ml, such as at a dose of 5-10 mg/ml, such as at a dose of 5 mg/ml of said composition. Commonly used doses are 2.5 mg/ml; 5 mg/ml; 7.5 mg/ml, 10 mg/ml; 20 mg/ml; 30 mg/ml; and is dependent on the specific anesthetic agent used as well as the specific conditions.

In one embodiment, the concentration of said alkanolamine in the composition is within the range of 0.1-20 M (mol/dm³), such as within the range of 0.2-18 M, such as within the range of 0.2-18 M, such as within the range of 0.5-15 M, such as within the range of 1-12 M, such as within the range of 2-10 M, such as within the range of 3-8 M, such as within the range of 4-6 M, such around 5 M.

In one embodiment, the concentration of said anesthetic agent in the composition is within the range of 0.1-500 mM (mol/dm³), such as within the range of 1-400 mM, such as within the range of 100-300 mM, such as within the range of 220-260 mM, such as within the range of 240-250 mM, such as around 245 mM.

In one embodiment, the concentration of PEG is around 0.1-100 μM, such as around 1-50 μM, such as around 5-15 μM, such as around 8 μM.

A composition of the invention may be prepared with different ratios of the components therein. For example, in one embodiment, there is a molar ratio of alkanolamine to anesthetic agent in the composition that is at least 2:1, such as at least 3:1, such as at least 4:1, such as at least 5:1, such as at least 6:1, such as at least 7:1, such as at least 8:1, such as at least 9:1, such as at least 10:1, such as at least 11:1, such as at least 12:1, such as at least 13:1, such as at least 14:1, such as at least 15:1, such as at least 16:1, such as at least 17:1, such as at least 18:1, such as at least 19:1, such as at least 20:1, such as at least 21:1, such as at least 22:1, such as at least 23:1, such as at least 24:1, such as at least 25:1.

A composition of the invention may be prepared with different pH values. For example, in one embodiment the pH of the composition is from pH 5 to pH 8, such as around pH 5, or such as around pH 5.5, or such as around pH 6, or such as around pH 6.5, or such as around pH 7, or such as around pH 7.5.

As discussed above, the present invention provides a formulation comprising at least one anesthetic agent selected from the group consisting of ropivacaine, bupivacaine, etidocaine, levobupivacaine, lidocaine, lignocaine, mepivacaine, articaine, dibucaine, levobupivacaine, prilocaine, benzocaine, chloroprocaine, cocaine, procaine, proparacaine, tetracaine and any pharmaceutically acceptable salt, hydrate, solvate or prodrug thereof.

In one embodiment, said least one anesthetic agent selected from the group consisting of ropivacaine, bupivacaine, etidocaine, levobupivacaine, lidocaine, lignocaine, mepivacaine, articaine, dibucaine, levobupivacaine, prilocaine, benzocaine, chloroprocaine, procaine, proparacaine, tetracaine and any pharmaceutically acceptable salt, hydrate, solvate or prodrug thereof.

In one embodiment, said least one anesthetic agent selected from the group consisting of ropivacaine, bupivacaine, etidocaine, levobupivacaine, lignocaine, mepivacaine, articaine, dibucaine, levobupivacaine, prilocaine, benzocaine, chloroprocaine, cocaine, procaine, proparacaine, tetracaine and any pharmaceutically acceptable salt, hydrate, solvate or prodrug thereof.

In one embodiment, said least one anesthetic agent selected from the group consisting of ropivacaine, bupivacaine, etidocaine, levobupivacaine, lidocaine, lignocaine, mepivacaine, articaine, dibucaine, levobupivacaine, prilocaine, chloroprocaine, cocaine, procaine, proparacaine and tetracaine.

In one embodiment, said least one anesthetic agent selected from the group consisting of ropivacaine, bupivacaine, etidocaine, levobupivacaine, lignocaine, mepivacaine, articaine, dibucaine, levobupivacaine, prilocaine, chloroprocaine, cocaine, procaine, proparacaine and tetracaine. In one embodiment, said least one anesthetic agent selected from the group consisting of ropivacaine, bupivacaine, etidocaine, levobupivacaine, lignocaine, mepivacaine, articaine, dibucaine, levobupivacaine, prilocaine, chloroprocaine, procaine, proparacaine and tetracaine.

In one embodiment, said least one anesthetic agent selected from the group consisting of ropivacaine, bupivacaine, etidocaine, levobupivacaine, lignocaine, mepivacaine and prilocaine. In one embodiment, said least one anesthetic agent selected from the group consisting of ropivacaine, bupivacaine, etidocaine, mepivacaine and prilocaine. In one embodiment, said least one anesthetic agent selected from the group consisting of ropivacaine, bupivacaine, mepivacaine and prilocaine. In one embodiment, said least one anesthetic agent selected from the group consisting of ropivacaine, bupivacaine and prilocaine. In one embodiment, said least one anesthetic agent selected from the group consisting of ropivacaine, bupivacaine and mepivacaine. In one embodiment, said least one anesthetic agent selected from the group consisting of ropivacaine, bupivacaine and etidocaine. In one embodiment, said anesthetic agent is selected from the group consisting of ropivacaine, bupivacaine and lidocaine. In one embodiment, said least one anesthetic agent is a combination of ropivacaine and lidocaine. In one embodiment, said least one anesthetic agent is a combination of bupivacaine and lidocaine. In one embodiment, said anesthetic agent is selected from the group consisting of ropivacaine and bupivacaine. In one embodiment, said anesthetic agent is bupivacaine. For the sake of clarity, the chemical formula of bupivacaine is presented as formula I below:

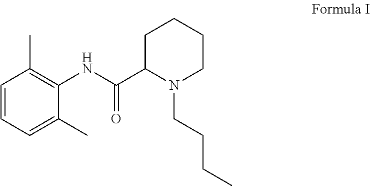 [SMILES: CCCCN1CCCCC1C(=O)Nc1c(C)cccc1C]

In one embodiment, said anesthetic agent is lidocaine. For the sake of clarity, the chemical formula of lidocaine is presented as formula II below:

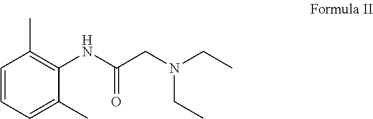 [SMILES: CCN(CC)CC(=O)Nc1c(C)cccc1C]

In one embodiment, said anesthetic agent is ropivacaine. For the sake of clarity, the chemical formula of ropivacaine is presented as formula III below:

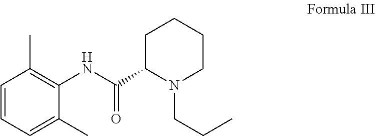 [SMILES: CCCN1CCCC[C@H]1C(=O)Nc1c(C)cccc1C]

In one embodiment, when said anesthetic agent is ropivacaine, said ropivacaine is in alkaline form. In one embodiment, said ropivacaine is present as a hydrochloride salt. Other pharmaceutically acceptable salts are also envisioned as suitable in the composition.

The skilled person appreciates that pharmaceutically acceptable salts of a anesthetic agent in accordance with this aspect of the present disclosure, are suitable to use in the composition. Salts which are suitable for use in medicine are those wherein a counterion is pharmaceutically acceptable. Examples of such pharmaceutically acceptable salts according to the invention include those formed with organic or inorganic acids or bases. In particular, suitable salts formed with acids according to the invention include those formed with mineral acids, strong organic carboxylic acids, such as alkane-carboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C1-C4)alkyl or aryl sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine.

In one embodiment, ropivacaine is present in its S-enantiomeric form. Preferably, S-ropivacaine may be present in enantiomeric excess. Such excess still allows R-ropivacaine to be present, however in a smaller amount compared to the S-form. The composition may comprise PEG. In one embodiment, said PEG is selected from the group consisting of PEG-3350, PEG-4000, PEG-6000, PEG-8000 and PEG-10000. In one embodiment, said PEG is PEG-3350 or PEG-4000. In one preferred embodiment, said PEG is PEG-4000.

The composition according to the present invention comprises water. The water may be purified water that is suitable for pharmaceutical applications. The water may be sterile water for injections (WFI). The water may be deionized water (diH₂O). The water may be distilled water (dH₂O). In one embodiment, said water is distilled water. Said distilled water may be MilliQ water. In one embodiment, said water is deionized water.

In one embodiment, the composition consists of:
ropivacaine;
triethanolamine;
water; and optionally
a pharmaceutically acceptable diluent, carrier and/or excipient.

In one embodiment, the composition consists of:
ropivacaine;
triethanolamine;
water;
PEG-4000; and optionally
a pharmaceutically acceptable diluent, carrier and/or excipient.

In one embodiment, there is a molar ratio of triethanolamine to ropivacaine in the composition that is at least 2:1, such as at least 3:1, such as at least 4:1, such as at least 5:1, such as at least 6:1, such as at least 7:1, such as at least 8:1, such as at least 9:1, such as at least 10:1, such as at least 11:1, such as at least 12:1, such as at least 13:1, such as at least 14:1, such as at least 15:1, such as at least 16:1, such as at least 17:1, such as at least 18:1, such as at least 19:1, such as at least 20:1, such as at least 21:1, such as at least 22:1, such as at least 23:1, such as at least 24:1, such as at least 25:1.

In one embodiment, the concentration of triethanolamine in the composition is within the range of 0.1-20 M (mol/dm³), such as within the range of 0.2-18 M, such as within the range of 0.2-18 M, such as within the range of 0.5-15 M, such as within the range of 1-12 M, such as within the range of 2-10 M, such as within the range of 3-8 M, such as within the range of 4-6 M, such around 5 M.

In one embodiment, the concentration of ropivacaine in the composition is within the range of 0.1-500 mM (mol/dm³), such as within the range of 1-400 mM, such as within the range of 100-300 mM, such as within the range of 220-260 mM, such as within the range of 240-250 mM, such as around 245 mM.

The composition may be suitable for storage, such as long-time storage. The composition may benefit from retained stability over time. In one embodiment, said composition remains stable upon storage for at least 1 day, such as for at least 1 week, such as for at least 2 weeks, such as for at least 3 weeks, such as for at least 4 weeks, such as for at least 5 weeks, such as for at least 6 weeks, such as for at least 7 weeks, such as for at least 8 weeks, such as for at least 9 weeks, such as for at least 10 weeks, such as for at least 3 months, such as for at least 6 months, such as for at least 1 year, such as for at least 2 years. For example, composition 1 and 2 of example 1 were subsequently used in example 3. Example 3 shows that the compositions were stable over a longer period of time and at different temperatures.

In one embodiment, said composition benefits from thermal stability. As such, said composition may remain thermally stable upon storage at a temperature of at least 4° C., such as at least 7° C., such as at least 10° C., such as at least 15° C., such as at least 20° C., such as at least 25° C., such as at least 30° C., such as at least 35° C., such as at least 40° C.

While stability may be determined in several ways, all apparent to a person of skill in the art, in one embodiment where the stability is retained, the stability is determined by a coefficient of variation. As used herein, the term stability is intended to mean that unwanted phenomena such as degradation of the components of the composition do not occur. As used herein, the phrase components of the composition is intended to include both the original components added into the composition as well as any components formed during the preparation of the composition. Stability is preferably determined by a coefficient of variation (“CV”). A skilled person realizes that a low measure of variation between samples (e.g. in a series of measurements over time, or over different temperatures) signifies a higher degree of retained stability. In one embodiment, “retained stability” means that the CV exhibited upon comparison of different samples is at most about 20%, such as at most 15%, such as at most 10%, such as at most 5%, such as at most 2.5%. As known to a person of skill in the art, other ways of measuring and denoting retained or maintained stability are also possible.

A composition intended for long-term storage may suitably be in the form of a freeze dried composition. As such, in one embodiment said composition is freeze dried. Upon freeze-drying a composition of the invention, water will be removed from the composition. It is however contemplated that a small portion of water is still present in the freeze dried composition.

It should be understood that the compositions according to the present disclosure may be useful as a therapeutic composition. A direct therapeutic effect, such as a long lasting anesthetic effect, may for example be accomplished by application of said composition.

In another aspect, there is provided a pharmaceutical composition according to the first aspect, for use in treating, alleviating or preventing pain by administration of said composition to a patient in need thereof. In one embodiment, said treating, alleviating or preventing pain provides for a long-lasting anesthesia, said anesthesia being for at least 0.5 hour, such as for at least 1 hour, such as for at least 2 hours, such as for at least 3 hours, such as for at least 4 hours, such as for at least 5 hours, such as for at least 6 hours, such as for at least 7 hours, such as for at least 8 hours, such as for at least 9 hours, such as for at least 10 hours, such as for at least 11 hours, such as for at least 12 hours, such as for at least 1 day, such for at least two days, such as for at least 3 days, such as for at least 4 days, such as for at least 5 days. An example of long-lasting pain relief using the composition of the invention is described in Examples 2 and 3. Another example of long-lasting pain relief using the composition of the invention is described in Example 6. Anesthesia may be measured in several ways. The skilled person appreciates that anesthesia may be measured both subjectively and objectively. For example, for a subjective measurement, a patient may be asked if a stimulus is hurting or providing another unpleasant feeling. Such a stimulus may be for example, a prick test, a pinch test, or said stimulus may result from a surgical procedure or a painful examination.

For example, for an objective measurement, vital parameters such as pulse, blood pressure and/or respiratory rate may be measured. This is common during surgical procedures. For an objective measurement, it is also possible to measure blockade of sensor or motor nerves by performing neurophysiological tests with surface electrodes on the skin, such as EMG (electromyography) for muscle activity (e.g. you ask the patient to lift the leg, bend the foot or the like), thereby muscle activity can measured.

The skilled person appreciates that the composition according to the invention is useful in treatment of all sorts of pain. In one embodiment, said pain is nociceptive pain or neuropathic pain. In one embodiment, said pain is caused by surgery, an injury or trauma. In one embodiment, said pain is postoperative pain, trauma pain or chronical pain.

In one embodiment, said use is in combination with an additional therapeutic agent. Suitable candidates for a combination treatment are apparent to a person of skill in the art and include for example the non-limiting list of antibacterial agents; antimycotic agents, antiviral agents, antiseptics, anti-inflammatory agents, antiphlogistics/analgesics (NSAID's) and antipruritic agents. For example, the additional therapeutically active agent may be selected from: antibacterial agents, such as oxytetracycline, fusidic acid, gentamycine, mupirocin, retapamulin (and pharmaceutically acceptable salts and derivatives thereof); antimycotic agents, such as nystatin, clotrimazole, miconazole, econazole, ketoconazole, bifonazole, and combinations of imidazole and triazole derivatives, ciclopirox, terbinafine, fluconazole, and amorolfine (and pharmaceutically acceptable salts and derivatives thereof); antiviral agents, such as aciclovir, valaciclovir, penciclovir, famciclovir, foscarnet (sodium phosphoneformate hexahydrate) and docosanol (and pharmaceutically acceptable salts and derivatives thereof); antiseptics, such as chlorhexidine, benzalkonium chloride and hydrogen peroxide; anti-inflammatory agents (glucocorticoids), such as hydrocortisone, clobetasone, triamcinolone, betamethasone, mometasone, desonide, prednisolone and clobetasol (and pharmaceutically acceptable salts and derivatives thereof); antiphlogistics/analgesics (NSAID's), such as acetylsalicylic acid, diclofenac, ketoprofen, ibuprofen, naproxen, capsaicin and nicotinate (and pharmaceutically acceptable salts and derivatives thereof); and antipruritic agents, such as glucocorticoids, for example, hydrocortisone, clobetasone, clobetasol, desonide, mometasone and betamethasone.

In one embodiment, said administration is at a dose of from about 0.1 to about 100 ml per kg body weight, such as from about 0.2 to about 50 ml per kg body weight, such as a dose of from about 0.3 to about 30 ml per kg body weight, such as a dose of from about 0.4 to about 20 ml per kg body weight, such as a dose of from about 0.5 to about 10 ml per kg body weight, such as a dose of from about 1 to about 5 ml per kg body weight of said composition.

In one embodiment, said administration is at a dose of from about 0.1 mg per kg body weight, such as from about 0.2 mg per kg body weight, such as a dose of from about 0.3 mg per kg body weight, such as a dose of from about 0.4 mg per kg body weight, such as a dose of from about 0.5 mg per kg body weight of said anesthetic, such as a dose of from about 0.6 mg per kg body weight of said anesthetic, such as a dose of from about 0.7 mg per kg body weight of said anesthetic, such as a dose of from about 0.75 mg per kg body weight of said anesthetic, such as a dose of from about 0.8 mg per kg body weight of said anesthetic, such as a dose of from about 0.9 mg per kg body weight of said anesthetic, such as a dose of from about 10 mg per kg body weight of said anesthetic.

In another aspect, there is provided a method of treating, alleviating or preventing pain, comprising administrating a therapeutically effective amount of a composition according to the first aspect to a patient in need thereof. In one embodiment, said pain is caused by surgery, an injury or trauma. In one embodiment, said method further comprises administration of an additional therapeutic agent.

Suitable additional therapeutic agents for a combination treatment are apparent to a person of skill in the art and include for example the non-limiting list of opioids and other opioid analogues, stimulants, antibacterial agents; antimycotic agents, antiviral agents, antiseptics, anti-inflammatory agents, antiphlogistics/analgesics (NSAID's) and antipruritic agents. For example, the therapeutically active agent of the invention may be selected from stimulants such as ephedrine; antibacterial agents, such as oxytetracycline, fusidic acid, gentamycine, mupirocin, retapamulin (and pharmaceutically acceptable salts and derivatives thereof); antimycotic agents, such as nystatin, clotrimazole, miconazole, econazole, ketoconazole, bifonazole, and combinations of imidazole and triazole derivatives, ciclopirox, terbinafine, fluconazole, and amorolfine (and pharmaceutically acceptable salts and derivatives thereof); antiviral agents, such as aciclovir, valaciclovir, penciclovir, famciclovir, foscarnet (sodium phosphoneformate hexahydrate) and docosanol (and pharmaceutically acceptable salts and derivatives thereof); antiseptics, such as chlorhexidine, benzalkonium chloride and hydrogen peroxide; anti-inflammatory agents (glucocorticoids), such as hydrocortisone, clobetasone, triamcinolone, betamethasone, mometasone, desonide, prednisolone and clobetasol (and pharmaceutically acceptable salts and derivatives thereof); antiphlogistics/analgesics (NSAID's), such as acetylsalicylic acid, diclofenac, ketoprofen, ibuprofen, naproxen, capsaicin and nicotinate (and pharmaceutically acceptable salts and derivatives thereof); and antipruritic agents, such as glucocorticoids, for example, hydrocortisone, clobetasone, clobetasol, desonide, mometasone and betamethasone.

In another aspect, there is provided a method for production of a composition comprising carbon quantum dots and at least one anesthetic agent selected from the group consisting of ropivacaine, bupivacaine, etidocaine, levobupivacaine, lidocaine, lignocaine, mepivacaine, articaine, dibucaine, levobupivacaine, prilocaine, benzocaine, chloroprocaine, cocaine, procaine, proparacaine, tetracaine and any pharmaceutically acceptable salt, hydrate, solvate or prodrug thereof; the method comprising:
a) bringing an alkanolamine selected from the group consisting of triethanolamine, tripropanolamine and trimethanolamine into contact with water, thereby obtaining a mixture;
b) heating said mixture, thereby obtaining carbon quantum dots;
c) adding at least one anesthetic agent selected from the group consisting of ropivacaine, bupivacaine, etidocaine, levobupivacaine, lidocaine, lignocaine, mepivacaine, and any pharmaceutically acceptable salt, hydrate, solvate or prodrug thereof to said mixture; wherein step b) and c) can be performed in any order;
d) optionally adding PEG to said mixture;
thereby obtaining a composition comprising carbon quantum dots and said anesthetic agent.

In one embodiment, said at least one anesthetic agent is selected from the list consisting of ropivacaine, bupivacaine, etidocaine, levobupivacaine, lidocaine, lignocaine, mepivacaine, articaine, dibucaine, levobupivacaine, prilocaine and any pharmaceutically acceptable salt, hydrate, solvate or prodrug thereof. In one embodiment, said at least one anesthetic agent is selected from the list consisting of ropivacaine, bupivacaine, etidocaine, levobupivacaine, lidocaine, lignocaine, mepivacaine, and any pharmaceutically acceptable salt, hydrate, solvate or prodrug thereof.

In one embodiment, step c) is preceding step b), which means that the mixture that is heated is the mixture comprising an alkanolamine, water and at least one anesthetic agent. In this embodiment, the mixture comprising an alkanolamine, water and at least one anesthetic agent forms carbon quantum dots.

In one embodiment, step b) is preceding step c), which means that the mixture that is heated is the mixture comprising an alkanolamine and water. In this embodiment, the mixture comprising an alkanolamine and water forms carbon quantum dots.

As shown in the appended examples, the pharmaceutical composition according to the present disclosure may be prepared by different routes. In one embodiment, an anesthetic agent, an alkanolamine and water are heated to form carbon quantum dots, with subsequent addition of PEG. This route is in analogy with Composition 1, described in Example 1.

In one embodiment, an alkanolamine and water are heated to form carbon quantum dots, with subsequent addition of an anesthetic agent, whereafter PEG is added.

This route is in analogy with Composition 2, described in Example 1. Example 2 of the present application show that both composition 1 and 2 of invention provides a longer duration of pain-relief, compared to the composition of the prior art.

In one embodiment, said heating is at a temperature of between 100° C. and 200° C., such as between 120° C. and 170° C., such as between 130° C. and 150° C., such as at 140° C. In one embodiment, said heating is by means of a microwave. Heating may also be applied by means of a heating plate or any other conventional heating means, as apparent to a person of skill in the art. In preferred embodiments, said heating is by means of a microwave oven.

Centrifuging may be useful for separating different components within the composition based on weight or on size, such as on particle weight or on particle size. Thus, in one embodiment said method further comprises a step of centrifuging said composition comprising carbon quantum dots and said anesthetic agent. In one embodiment, said centrifugation is at a speed of 2000-5000 rpm, such as at 4000 rpm. In one embodiment, said centrifugation is for at least 5 minutes, such as for at least 10 minutes. The skilled person will appreciate suitable conditions for centrifugation.

In another aspect, there is provided a carbon quantum dot comprising an anesthetic agent selected from the group consisting of ropivacaine, bupivacaine, etidocaine, levobupivacaine, lidocaine, lignocaine, mepivacaine, and any pharmaceutically acceptable salt, hydrate, solvate or prodrug thereof obtainable by the method for production of a composition comprising carbon quantum dots and at least one anesthetic agent, in accordance with the aspects described above.

In one embodiment, said at least one anesthetic agent is selected from the list consisting of ropivacaine, bupivacaine, etidocaine, levobupivacaine, lidocaine, lignocaine, mepivacaine, articaine, dibucaine, levobupivacaine, prilocaine and any pharmaceutically acceptable salt, hydrate, solvate or prodrug thereof. In one embodiment, said at least one anesthetic agent is selected from the list consisting of ropivacaine, bupivacaine, etidocaine, levobupivacaine, lidocaine, lignocaine, mepivacaine, and any pharmaceutically acceptable salt, hydrate, solvate or prodrug thereof.

In one embodiment, said anesthetic agent is selected from the group consisting of ropivacaine, bupivacaine and lidocaine. In one embodiment, said anesthetic agent is ropivacaine. In one embodiment, said alkanolamine is triethanolamine. In one embodiment, the carbon quantum dot further comprises PEG, optionally wherein said PEG is selected from the group consisting of PEG-3350, PEG-4000, PEG-6000, PEG-8000 and PEG-10000. In one embodiment, said PEG is PEG-4000.

In one embodiment, said dot is fluorescent. Said dot may emit light at a wavelength of from 400 to 600 nm, such as at wavelength of from 415 to 430 nm, such as at a wavelength of around 425 nm. In one embodiment, said dot has an average diameter of between 1 and 20 nm, such as from 2 to 18 nm, such as from 3 to 15 nm, such as from 4 to 12 nm, such as from 5 to 10 nm. Under UV-light, said carbon quantum dot may appear blue by ocular inspection.

In yet another aspect, there is provided a carbon quantum dot formed from a composition comprising:
at least one anesthetic agent selected from the group consisting of ropivacaine, bupivacaine, etidocaine, levobupivacaine, lidocaine, lignocaine, mepivacaine, articaine, dibucaine, levobupivacaine, prilocaine, benzocaine, chloroprocaine, cocaine, procaine, proparacaine, tetracaine and any pharmaceutically acceptable salt, hydrate, solvate or prodrug thereof;
at least one alkanolamine selected from the group consisting of triethanolamine, tripropanolamine and trimethanolamine; and
water.

In one embodiment, said at least one anesthetic agent is selected from the list consisting of ropivacaine, bupivacaine, etidocaine, levobupivacaine, lidocaine, lignocaine, mepivacaine, articaine, dibucaine, levobupivacaine, prilocaine and any pharmaceutically acceptable salt, hydrate, solvate or prodrug thereof. In one embodiment, said at least one anesthetic agent is selected from the list consisting of ropivacaine, bupivacaine, etidocaine, levobupivacaine, lidocaine, lignocaine, mepivacaine, and any pharmaceutically acceptable salt, hydrate, solvate or prodrug thereof.

In one embodiment, said dot is fluorescent. Said dot may emit light at a wavelength of from 400 to 600 nm, such as at wavelength of from 415 to 430 nm, such as at a wavelength of around 425 nm. In one embodiment, said dot has an average diameter of from 1 and 20 nm, such as of from 2 to 18 nm, such as form 3 to 15 nm, such as from 4 to 12 nm, such as from 5 to 10 nm. Under UV-light, said carbon quantum dot may appear blue by ocular inspection.

In a related aspect, there is provided a use of a composition according to the aspects described above for the manufacture of a medicament for use in the treatment of pain.

### Definitions

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.” In case of conflict, the present specification, including explanations of terms, will control over any other source of definition. In addition, all the materials, methods, and examples are illustrative and not intended to be limiting.

The term “pharmaceutical composition” is used in its widest sense, encompassing all pharmaceutically applicable compositions containing at least one active substance and optional carriers, adjuvants, diluents, constituents etc. The term “pharmaceutical composition” also encompasses a composition comprising the active substance in the form of derivate or a pro-drug, such as pharmaceutically acceptable salts, sulphates and esters. The manufacture of pharmaceutical compositions for different routes of administration falls within the capabilities of a person skilled in galenic chemistry.

The term “prodrug” is used herein to describe a compound that participates in a chemical reaction to form an anesthetic agent in accordance with the present invention.

The terms “administration” and “mode of administration” as well as “route of administration” are also used in their widest sense. The pharmaceutical composition of the present invention may be administered in a number of ways depending largely on whether a local or topical mode of administration is most appropriate for the specific pain be treated. These different modes of administration are for example topical (e.g., on the skin), local (including ophthalmic and to various mucous membranes, for example vaginal and rectal delivery), oral or parenteral. The preparation of such compositions and formulations is generally known to those persons skilled in formulation arts and may be applied to the formulation of the composition of the present invention.

Typical single dosage compositions (or unit dosage compositions) are those containing an effective dose, as hereinbefore recited, or an appropriate fraction thereof, of the active ingredient.

It should be understood that in addition to the ingredients particularly mentioned above, the compositions of this invention may include other agents conventional in the art having regard to the type of formulation in question.

The terms “composition” and “formulation” are used interchangeably throughout the application, and are intended have the same meaning, as apparent to a person of skill in the art.

The terms “subject” and “patient” are used interchangeably throughout the application, and are intended have the same meaning, as apparent to a person of skill in the art.

As used herein, when the term “about” or “approximately” is used in relation to a numerical value, it is to be interpreted as a range of ±10%, such as ±9%, such as ±8%, such as ±7%, such as ±6%, such as ±5%, such as ±4%, such as ±3%, such as ±2%, such as ±1%. For example, when the value is stated to be about 10, this means that the value is in fact in the range of from 9 to 11, such as in the range of from 9.9 to 10.9, such as in the range of from 9.8 to 10.8, such as in the range of from 9.7 to 10.7, such as in the range of from 9.6 to 10.6, such as in the range of from 9.5 to 10.5, such as in the range of from 9.4 to 10.4, such as in the range of from 9.3 to 10.3, such as in the range of from 9.2 to 10.2, such as in the range of from 9.1 to 10.1.

The skilled person knows that numerical values relating to measurements are subject to measurement errors which place limits on their accuracy. For this reason, the general convention in the scientific and technical literature is applied: the last decimal place of a numerical value indicates its degree of accuracy. Where no other error margins are given, the maximum margin is ascertained by applying the rounding-off convention to the last decimal place e.g. for a measurement of 3.5 cm, the error margin is 3.45-3.54. When interpreting ranges of values in patent specifications, the skilled person proceeds on the same basis.

As apparent to a person of skill in the art, the term tripropanolamine comprises tri-iso-propanoleamin.

While the invention has been described with reference to various exemplary aspects and embodiments, it will be understood by those skilled in the art that various changes may be made and equivalents may be substituted for elements thereof without departing from the scope of the invention. Therefore, it is intended that the invention is not limited to any particular embodiment contemplated, but that the invention will include all embodiments falling within the scope of the appended claims. The invention will be further illustrated by the following non-limiting Examples.

### BRIEF DESCRIPTION OF THE DRAWINGS

FIG. 1 shows a fluorescence spectrum. The samples were prepared according to Example 1 (compositions 1-3; which are denoted test 1-3).

FIG. 2 shows a fluorescence spectrum (average of 3 readings). The samples were prepared according to Example 4, samples 3 and sample 4 (see table 10).

FIG. 3 shows a fluorescence spectrum (average of 3 readings). The samples were prepared according to Example 4, sample 5 (see table 10).

### REFERENCES

Kuthiala and Chaudhary. Ropivacaine: A review of its pharmacology and clinical use. Indian J Anesth 2011; 55: 104-10.
Qu et al. Synthesis of bifunctional carbon quantum dots for bioimaging and anti-inflammation. Nanotechnology 2020; 31. 175102

### EXAMPLES

### Example 1: Characterization of Quantum Dots

### Material and Methods

Ropivacaine was purchased from AK Scientific (purity 98%). Triethanolamine was purchased from Chemtronica. PEG4000 was purchased from Chemtronica. The microwave oven used was a Milestone MLS 1200 Pyro High temperature muffle furnace with temperature control.

### Preparation of Compositions 1-3

### Composition 1—Composition Consisting of Ropivacaine, Triethanolamine, Water and PEG400

1. Ropivacaine free base (0.4 g) was mixed with purified water (2 mL) and Triethanolamine (4 mL).
2. The mixture from step 1 at was heat treated at 140° C. for 20 minutes in a microwave oven.
3. A 0.5 mL sample was taken for fluorescence and particle size measurements.
4. PEG4000 (0.2 mg) was added.
5. A 0.5 mL sample was taken for fluorescence and particle size measurements.

### Observations:

Not a clear solution at any step.

### Composition 2—Composition Consisting of Ropivacaine, Triethanolamine, Water and PEG400

1. Purified water (2 mL) and Triethanolamine (4 mL) was mixed.
2. The mixture from step 1 at was heat treated at 140° C. for 20 minutes in a microwave oven.
3. A 0.5 mL sample was taken for fluorescence and particle size measurements.
4. Ropivacaine free base (0.4 g) was added to the mixture of step 2.
5. PEG4000 (0.2 mg) was added.
6. A 0.5 mL sample was taken for fluorescence and particle size measurements.

### Observations:

Not a clear solution at step 4

### Composition 3—Composition Consisting of Ropivacaine, Water and PEG400

1. Ropivacaine free base (0.4 g) was mixed with purified water (2 mL).
2. The mixture from step 1 at was heat treated at 140° C. for 20 minutes in a microwave oven.
3. A 0.5 mL sample was taken for fluorescence and particle size measurements.
4. PEG4000 (0.2 mg) was added.
5. A 0.5 mL sample was taken for fluorescence and particle size measurements.

### Observations:

Not a clear solution at any step.

### Solubility

An ocular inspection of solubility was done. The results thereof are shown in Table 1.

TABLE 1

| Test # | Before microwave | After microwave | After addition of PEG |
|---|---|---|---|
| 1 | cloudy | cloudy | cloudy/partly clear |
| 2 | clear | clear (beige) | partly cloudy (beige) |
| 3 | 2-phase | 2-phase | 2-phase/partly cloudy |

### Fluorescence Measurements

Fluorescence was measured on a microplate reader (Thermo Scientific Varioscan LUX SN 3020-836, REF VLBL001D1, Type 3020). A 0.5 mL sample was taken for the measurements, according to the method described for composition 1, 2 and 3. The samples were prepared by centrifuge filtration through a 0.45 μm filter. Measurements were taken as duplicates. Emission wavelength used was 400-700 nm and excitation wavelength used was 350 nm. Absorbance (AU) was measured.

TABLE 2

| | | Max | Max. |
|---|---|---|---|
| | | intensity | intensity |
| Entry | | A | B |
| A | Composition 1 before addition of PEG | 4.022 | 3.868 |
| B | Composition 1 after addition of PEG | 3.863 | 3.4008 |
| C | Composition 2 before addition of | 21.66 | 20.47 |
| | ropivacaine and PEG | | |
| D | Composition 2 after addition of | 15.55 | 8.850 |
| | ropivacaine and PEG | | |
| E | Composition 3 before addition of PEG | 0.8577 | 0.8725 |
| F | Composition 3 after addition of PEG | 1.251 | 1.035 |

### Results

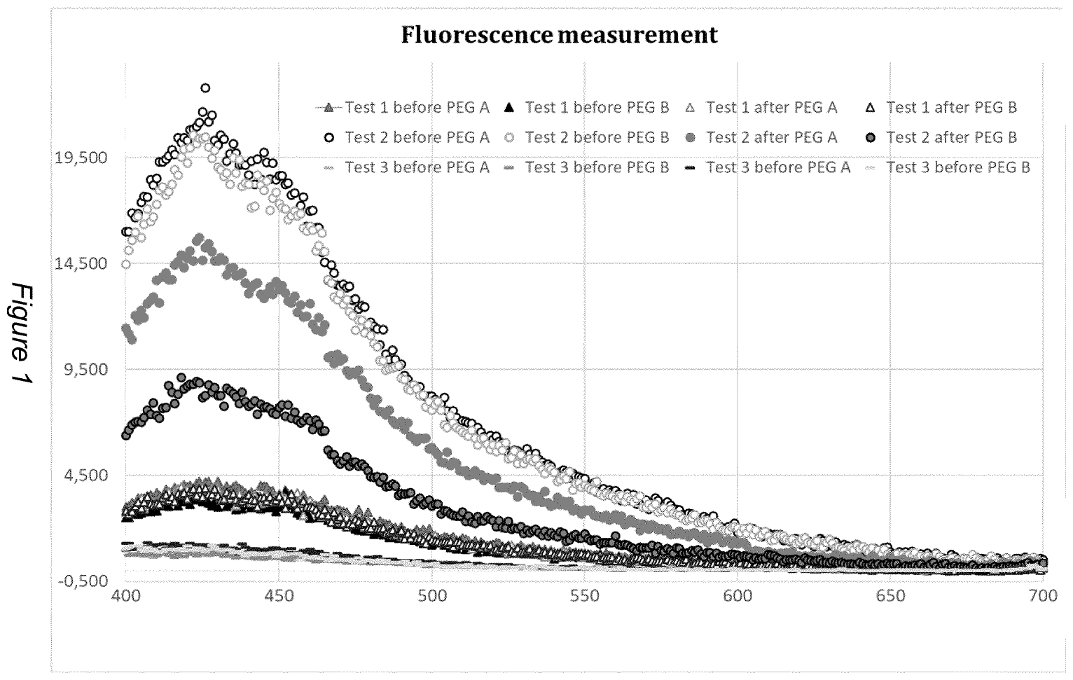

Fluorescence was essentially blue. The highest intensity of fluorescence was found for Composition 2, and was highest before addition of PEG. PEG seemed to lower fluorescence. The samples of Composition 1 did also show significant fluorescence, both before and after addition of PEG. Essentially no fluorescence was seen in Composition 3. A spectrum of the fluorescence is found in FIG. 1.

The following was noted it relation to entries A-F:

A and B, which correspond to the composition in which ropivacaine, water and triethanolamine were heated in the microwave, with subsequent addition of PEG for entry B; show high fluorescence with a slight intensity decrease in fluorescence after addition of PEG.

C and D, which correspond to the composition in which water and triethanolamine were heated in the microwave with addition of ropivacaine after heat treatment, with subsequent addition of PEG for entry D; show significant fluorescence with a slight intensity decrease in fluorescence after addition of PEG.

E and F, which corresponds to the composition in which only ropivacaine was heated in the microwave, with subsequent addition of PEG, show low fluorescence.

### Particle Size Measurements

Average Particle size distribution by intensity (Z-ave) was measured with Dynamic Light Scattering on a Zetasizer Nano ZS from Malvern Instruments. A 0.5 mL sample was taken for the measurements, according to composition 1, 2 and 3. The samples were prepared by centrifuge filtration through a 0.45 μm filter. Measurements were taken as duplicates.

PDI: A dimensionless value (PolyDistribution Index) of the width of the distribution. If <0.3 the distribution is normally considered mono disperse (monomodal). As can be seen from Table 3, all samples but Entry E are considered monomodal.

TABLE 3

| Entry | | Z-Ave [nm] | PDI |
|---|---|---|---|
| A | Composition 1 before addition of PEG | 5.382 | 0.234 |
| B | Composition 1 after addition of PEG | 5.838 | 0.247 |
| C | Composition 2 before addition of API | 9.276 | 0.244 |
| | and PEG | | |
| D | Composition 2 after addition of API | 7.399 | 0.237 |
| | and PEG | | |
| E | Composition 3 before addition of PEG | 132.9 | 0.345 |
| F | Composition 3 after addition of PEG | 3.77 | 0.16 |

Results are an average of duplicate measurements

The particle size (Z-Ave) is in line to what is expected of a blue fluorescence carbon quantum dot.

### CONCLUSIONS

Composition 1 does comprise carbon quantum dots, which indicates that such dots can be formed from triethanolamine, ropivacaine and water when heat is applied. It is plausible that ropivacaine can be changed to any anesthetic agent in accordance with the present invention, and will provide the same of similar result.

Composition 2 does comprise carbon quantum dots, which indicates that such dots can be formed from triethanolamine and water when heat is applied. It is plausible that ropivacaine can be changed to any anesthetic agent in accordance with the present invention, and will provide the same of similar result.

Composition 3 does not comprise carbon quantum dots, which indicates that such dots cannot be formed from ropivacaine and water.

Without being bound by any theory, the fluorescence and small particle size is believed to be due to the formation of carbon quantum dots, which are formed during the microwave heat treatment of triethanolamine. The modified formulation was also tested in-vivo in an animal model to assess duration of local anesthesia, see examples 2 and 3.

### Example 2: In Vivo Studies

In order to evaluate administration of local anesthesia in a sensitivity test in female rats a pilot- and an efficacy study was performed.

The experiment was performed at Redoxis AB, Lund (Sweden). The animals were maintained in modern, approved animal facilities to assure well-being of the animals. All experiments performed by Redoxis had an ethical permit and followed Swedish national laws.

Sprauge Dawley rats (females, 10 weeks) were acquired from Janvier Europe. Rats were housed in the animal facility, Medicon Village, Lund, Sweden. The rats were kept at 12 h light/dark cycles in polystyrene cages (type III cages, 2-3 rats per cage) comprising wood shavings. The rats were fed with standard rodent diet and water ad libitum. The rats were acclimatized for approximately one week before initiation of the experiment. The rats were identified by a marking on the tail with a permanent marker day 1.

### Local Anesthesia Composition—Pilot Study

TABLE 4

| | |
|---|---|
| Number of animals | 6 females (Sprague Dawly, age 8 weeks) |
| Group size | 3 rats/group |
| Drug administration | One s.c. injection |
| Prior art formulation | Ropivacaine free base, 0.5 mg/mL |
| 0.5 mg/mL of the free base | |
| Study duration | 6 hours |
| Evaluation | Prick test |
| Groups | 1) Vehicle |
| | 2)Reference local anesthesia Ropivacaine |

### Group Composition

The rats were weighed day −1 (the day before the experiment started) in order to determine the mean weight of the animals included in the experiment.

### Compound and Administration

Formulation of reference anesthesia ropivacaine, was prepared such that the pH of the composition and the concentration and dose of ropivacaine where essentially identical to the compositions of the invention. The concentration of Ropivacaine was 5 mg/ml in the formulation and was administered at a dose of 1 mg/kg bodyweight in 2 animals, 0.25 mg/kg bodyweight in 2 animals; and placebo PBS (phosphate buffered saline) in 2 animals. The vehicle and reference anesthesia were administrated s. c. (subcutaneous) in one single injection at time 0.

### Sensitivity Test and Evaluation

The rats were shaved at the back and habituated to the experimental environment for 30 minutes prior to the testing. The sensitivity test was performed at 5 different time points within 5 hours after administration of the test item (30 min, 1 h, 2 h, 3 h, 4 h, 5 h) by touching each rat with a needle. The rat's reaction in the form of wrinkling of the skin was evaluated by ocular inspection.

### Health Assessment

Macroscopic evaluation: rats were monitored for assessment of health throughout the study. Any adverse effect would have been observed according to health assessment protocol. In the event of a rat having bad health (such as for example a rat with problem with dehydration or kyphotic posture), such a rat would have been removed from the experiment. However, all rats used in this experiment stayed healthy.

### Results

The results showed that the design of the study was suitable for testing efficacy of ropivacaine. The results (data not shown) showed that the group receiving ropivacaine at a dose of 0.25 mg/kg bodyweight had a duration of the anesthetic effect of 2 h, and that the group receiving ropivacaine at a dose of 1 mg/kg bodyweight had a duration of the anesthetic effect of 5 h. The placebo group had no anesthetic effect.

### Local Anesthesia Test—Efficacy Test

TABLE 5

| | |
|---|---|
| Number of animals | 12 females (Sprague Dawly, age 8 weeks) |
| Group size | 3 rats/group |
| Drug administration | One s.c. injection |
| Formulation (0.5 mg/mL | According to Example 1, composition 1 |
| of the free base) | (group 3) or 2 (group 4) |
| Study duration | 5 hours |
| Evaluation | Prick test |
| Groups | 1) Vehicle |
| | 2)Reference local anesthesia Ropivacaine |
| | 3) Composition 1 formulation (as prepared |
| | in example 1) |
| | 4) Composition 2 formulation (as prepared |
| | in example 1) |

### Group Composition

The rats were weighed day −1 (the day before the experiment started) in order to determine the mean weight of the animals included in the experiment.

### Compound and Administration

Formulation of reference anesthesia Ropivacaine, was prepared in the same way as for the pilot study. The concentration of Ropivacaine (both in the reference and in composition 1 and composition 2) was 0.5 mg/ml in the formulation and was administered at a dose of 0.25 mg/kg body weight. The vehicle, reference anesthesia, composition 1 anesthesia and composition 2 anesthesia were administrated s. c. in one single injection at time 0.

### Sensitivity Test and Evaluation

The rats were shaved at the back and habituated to the experimental environment for 30 minutes prior to the testing. The sensitivity test was performed at 6 different time points within 6 hours after administration of the test item (30 min, 1 h, 2 h, 3 h, 4 h, 5 h) by touching each rat with a needle. The rat's reaction in the form of wrinkling of the skin was evaluated by ocular inspection, giving each monitored response a value of 0 (no response) or 1 (response). No response demonstrations that the rat experienced anesthesia.

### Health Assessment

Macroscopic evaluation: rats were monitored for assessment of health throughout the study. Any adverse effect would have been observed according to health assessment protocol. In the event of a rat having bad health (such as for example a rat with problem with dehydration or kyphotic posture), such a rat would have been removed from the experiment. However, all rats used in this experiment stayed healthy.

### Results

TABLE 6
Results from the prick test. Group 1 received placebo, group 2
received ropivacaine, group 3 received a composition according
to the present invention (composition 1), group 4 received a
composition according to the present invention (composition 2).

| Group | ID | 0.5 h | 1 h | 2 h | 3 h | 4 h | 5 h |
|---|---|---|---|---|---|---|---|
| 1 | B2 | 0 | 1 | 0 | 1 | 1 | 1 |
| 1 | C3 | 1 | 1 | 0 | 1 | 1 | 1 |
| 1 | D3 | 0 | 0 | 0 | 0 | 0 | 1 |
| 2 | C2 | 0 | 0 | 1 | 1 | 1 | 1 |
| 2 | B3 | 1 | 1 | 1 | 1 | 1 | 1 |
| 2 | A1 | 0 | 0 | 0 | 1 | 0 | 1 |
| 3 | A3 | 0 | 0 | 0 | 0 | 1 | 1 |
| 3 | C1 | 0 | 0 | 0 | 0 | 1 | 1 |
| 3 | D1 | 0 | 0 | 0 | 0 | 0 | 1 |
| 4 | A2 | 0 | 0 | 0 | 0 | 1 | 1 |
| 4 | B1 | 0 | 0 | 0 | 0 | 1 | 1 |
| 4 | D2 | 1 | 1 | 1 | 1 | 1 | 1 |

The results can be presented as the average value of the first response. As evident from table 6, the compositions according to the invention benefit from an increase in anesthesia duration. Compared to the prior art (group 2), group 3 had an increase in duration of approximately 2.4×(2.4 times longer duration of anesthetic effect) and group 4 had an increase in duration of approximately 1.6×(1.6 times longer duration of anesthetic effect).

TABLE 7
Average value of first response.

| Group | Average value of first response (h) |
|---|---|
| 2 | 1.8 |
| 3 | 4.3 |
| 4 | 2.8 |

### CONCLUSION

Composition 1 (used in group 3) does comprise carbon quantum dots, which indicates that such dots provide an increase in pain-relief duration in vivo. It is plausible that ropivacaine can be changed to any anesthetic agent in accordance with the present invention, and will provide the same of similar in vivo effect.

Composition 2 (used in group 4) does comprise carbon quantum dots, which indicates that such dots provide an increase in pain-relief duration in vivo. It is plausible that ropivacaine can be changed to any anesthetic agent in accordance with the present invention, and will provide the same of similar in vivo effect.

### Experiment 3: In Vivo Test 3

TABLE 8

| | |
|---|---|
| Number of animals | 12 rats |
| Group size | 6 or 4 rats/group |
| Drug administration | One s.c. injection |
| Formulation (0.5 mg/mL | According to Example 1, composition 1 |
| of the free base) | (group 3) or 2 (group 4) |
| Study duration | 9 hours |
| Evaluation | Sensitivity test |
| Groups | 1)Reference local anesthesia Ropivacaine |
| | 2) Composition 1 formulation (as prepared |
| | in example 1) |
| | 3) Composition 2 formulation (as prepared |
| | in example 1) |

### Animals

Male Wistar albino rats, weighing 200-250 g, obtained from SYLAB Experimental Animals Laboratory, were used in the experiments. The animals were kept in a 12 hour light-12 hour dark light period under standard conditions and fed with standard pellet water (ad libitum). All studies were carried out in accordance with international ethical rules, without abusing animal rights.

### Compound and Administration

Formulations used were the same as in Example 2, with additional storage. The formulations were stored for 7 months at different temperatures. At first, the formulations were stored in the freezer for 6 months. Thereafter there were alterations in temperature storage; the formulations were iteratively stored in room temperature and in the fridge before use.

The compositions were administrated s. c. in one single injection at time 0. The compositions were administrated to a position in close proximity to the sciatic nerve.
Group 1: Ropivacaine 7.5 mg/ml, 1.1-1.2 ml. Total=at least 8.25 mg ropivacaine. This composition is commercial ropivacaine (Ropivacaina Inibsa, injectable solution).
Group 2: Ropivacaine 4 mg/ml, 2 ml, pH 5 (Test 1). Total=8 mg ropivacaine. This composition is composition 1 in example 1.
Group 3: Ropivacaine 4 mg/ml, 2 ml, pH 5 (Test 2). This composition is composition 2 in example 1.

### Sensitivity Test and Evaluation

The sensitivity test was performed at 9 different time points within 9 hours after administration of the test item (1 h, 1 h, 2 h, 3 h, 4 h, 5 h, 6 h, 7 h, 8 h and 9 h) by touching each rat with a tweezer. The rat's reaction in the form of wrinkling of the skin was evaluated by ocular inspection, giving each monitored response a value of 0 (no response), 0.5 (low response) or 1 (response). No response demonstrations that the rat experienced anesthesia. Low response demonstrations that the rat partly experienced anesthesia.

### Results

Table 8 a-c. Results from the sensitivity test. Group 1 received commercially available ropivacaineformulation, group 2 received a composition according to the present invention (composition 1), group 3 received a composition according to the present invention (composition 2).

TABLE 8a

| Test time | | Group I: Ropivacaine | | | | | |
|---|---|---|---|---|---|---|---|
| [h] | | Rat 1 | Rat 2 | Rat 3 | Rat 4 | Rat 5 | Rat 6 |
| 0 | Motor | 1 | 0.5 | 1 | 1 | 1 | 1 |
| | Block: | | | | | | |
| | Sensor | 1 | 1 | 1 | 1 | 1 | 1 |
| | Block: | | | | | | |
| 1 | M: | 1 | 0.5 | 1 | 1 | 1 | 1 |
| | S: | 1 | 1 | 1 | 1 | 1 | 1 |
| 2 | M: | 1 | 0.5 | 1 | 1 | 0.5 | 1 |
| | S: | 1 | 1 | 1 | 1 | 1 | 1 |
| 3 | M: | 0.5 | 0 | 0 | 1 | 0 | 1 |
| | S: | 1 | 0 | 0 | 1 | 1 | 1 |
| 4 | M: | 0 | 0 | 0 | 0 | 0 | 0 |
| | S: | 1 | 0 | 0 | 0 | 0.5 | 0 |
| 5 | M: | 0 | | | 0 | 0 | 0 |
| | S: | 1 | | | 0 | 0.5 | 0 |
| 6 | M: | 0 | | | | 0 | |
| | S: | 0 | | | | 0 | |
| 7 | M: | 0 | | | | 0 | |
| | S: | 0 | | | | 0 | |
| 8 | M: | | | | | | |
| | S: | | | | | | |
| 9 | M: | | | | | | |
| | S: | | | | | | |

| | | | | | | |
|---|---|---|---|---|---|---|
| Loss Motor block (h) | 4 | 3 | 3 | 4 | 4 | 4 |
| average (h) | 3.3 | | | | | |
| loss of Sensor block | 6 | 3 | 3 | 4 | 6 | 4 |
| average (h) | 4.3 | | | | | |

TABLE 8b

| | |
|---|---|
| Test time | Group II: 22E1552-1 |

| [h] | | Rat 7 | Rat 8 | Rat 9 | Rat 14 |
|---|---|---|---|---|---|
| 0 | Motor | 1 | 1 | 1 | 1 |
| | Block: | | | | |
| | Sensor | 1 | 1 | 1 | 1 |
| | Block: | | | | |
| 1 | M: | 1 | 1 | 1 | 1 |
| | S: | 1 | 1 | 1 | 1 |
| 2 | M: | 1 | 1 | 1 | 1 |
| | S: | 1 | 1 | 1 | 1 |
| 3 | M: | 1 | 1 | 1 | 1 |
| | S: | 1 | 1 | 1 | 1 |
| 4 | M: | 1 | 1 | 1 | 1 |
| | S: | 1 | 1 | 1 | 1 |
| 5 | M: | 1 | 1 | 0 | 1 |
| | S: | 1 | 1 | 0 | 1 |
| 6 | M: | 1 | 1 | 0 | 0 |
| | S: | 1 | 1 | 0 | 0 |
| 7 | M: | 0.5 | 0 | | 0 |
| | S: | 1 | 1 | | 0 |
| 8 | M: | 0 | 0 | | |
| | S: | 0.5 | 0 | | |
| 9 | M: | 0 | 0 | | |
| | S: | 0.5 | 0 | | |

| | | | | |
|---|---|---|---|---|
| Loss Motor block (h) | 8 | 7 | 5 | 6 |
| average (h) | 6.5 | | | |
| loss of Sensor block | 10 | 8 | 5 | 6 |
| average hours | 7.25 | | | |

TABLE 8c

| Test time | | Group III: 22E1552-1 | | | |
|---|---|---|---|---|---|
| [h] | | Rat 10 | Rat 11 | Rat 12 | Rat 13 |
| 0 | Motor | 0 | 1 | 1 | 1 |
| | Block: | | | | |
| | Sensor | 1 | 1 | 1 | 1 |
| | Block: | | | | |
| 1 | M: | 0 | 1 | 1 | 1 |
| | S: | 1 | 1 | 1 | 1 |
| 2 | M: | 0 | 1 | 1 | 1 |
| | S: | 1 | 1 | 1 | 1 |
| 3 | M: | 0 | 1 | 1 | 1 |
| | S: | 0 | 1 | 1 | 1 |
| 4 | M: | 0 | 1 | 1 | 1 |
| | S: | 0 | 1 | 1 | 1 |
| 5 | M: | | 1 | 1 | 1 |
| | S: | | 1 | 1 | 1 |
| 6 | M: | | 1 | 0 | 1 |
| | S: | | 1 | 0 | 1 |
| 7 | M: | | 0.5 | 0 | 0 |
| | S: | | 1 | 0 | 1 |
| 8 | M: | | 0 | | 0 |
| | S: | | 0 | | 0 |
| 9 | M: | | 0 | | 0 |
| | S: | | 0 | | 0 |

| | | | | |
|---|---|---|---|---|
| Loss Motor block (h) | 0 | 8 | 6 | 7 |
| average (h) | 5.25 | | | |
| loss of Sensor block | 3 | 8 | 6 | 8 |
| average hours | 6 | | | |

### DISCUSSION

For two rats in the experiment, the syringe was applied at the wrong position, i.e. either into the nerve itself (rat 1) or not in close proximity to the sciatic nerve (rat 2). This was due to human error and sudden movement of the rat.

Rat 1 experienced spasm, which is believed to be due to that the composition was erroneously given into the sciatic nerve, causing paralysis. This administration of the composition may have caused an increase in anesthetic effect, which is an evidence based side effect of wrongly administration in to the nerve. As shown in Table 8a, rat 1 had an increased effect, which is not due to the prior art composition as such, but rather to said side effect

At the time of administration of the local anesthetic composition, rat 10 unexpectedly jumped. This caused the injection to be administrated at the wrong site of the rat, far away from the sciatic nerve. As can be seen in Table 8c, Rat 10 did not sense any anesthetic effect at all. It is believed that the reason for not experiencing any anesthetic effect is due to administration at the wrong site of the rat.

### CONCLUSION

Composition 1 (used in group 2) does comprise carbon quantum dots, which indicates that such dots provide an increase in pain-relief duration in vivo. It is plausible that ropivacaine can be changed to any anesthetic agent in accordance with the present invention, and will provide the same of similar in vivo effect.

Composition 2 (used in group 3) does comprise carbon quantum dots, which indicates that such dots provide an increase in pain-relief duration in vivo. It is plausible that ropivacaine can be changed to any anesthetic agent in accordance with the present invention, and will provide the same of similar in vivo effect.

When comparing tables 8a, 8b and 8c, it is evident that the composition according to the present invention (table 8b and 8c) provides a longer anesthetic effect compared to the composition of the prior art (table 8a).

### Example 4: Further Characterization of the Formulation

The aim of this work was to assess if and to which degree the triethanolamine is converted into something else during the microwave heat treatment and if this treatment would create anything that could influence the stability of ropivacaine.

### Materials

TABLE 9
Materials used

| Component | Source | Lot/Quality |
|---|---|---|
| Ropivacaine | Chemtronica/ AK | LC56464 / 98% (GC) |
| | Scientific | |
| Triethanol | Merck, Emprove | K49251272 / 102% |
| amine | Essential | (NMR-Assay) |
| PEG 4000 | Merck, Emprove | K54052306 |
| | Essential | |
| Water | RISE | ASTM Type 1 water |
| 2,6-Dimethylaniline | Alfa Aesar | 10193839 / 99% |

### Methods

### Heat Treatment of Triethanolamine

Triethanolamine (TEA) was mixed with water in the volume ratio 2 parts triethanolamine and 1 part water. This mixture was then heat treated in a microwave oven at 140° C./500 W for 20 minutes (High temp muffle furnace mis 1200 pyro). The oven was pre-heated to 140° C. before samples were placed in the oven. Before samples were taken out of the oven the temperature was allowed to decrease to 100° C., a process which took approximately an additional 10 minutes.

### Preparation of the Final Formulation

To 3 mL of the heat treated triethanolamine/water mixture 200 mg of ropivacaine free base and 100 mL PEG 4000 is added. The mixture was diluted with water to approx. 90% of its final volume and the pH is adjusted to approx. 5.0 which yields a clear solution.

Also, a version without PEG 4000 has been used in the assessment.

### Fluorescence Measurements

Fluorescence was measured using a fluorometer, VARIOSKAN LUX, Thermo Scientific, with plate reader. For the measurement 100 or 300 μL of the sample according to 3.1.2 was added to a black 96 wells plate. Excitation wavelength was selected to 350 nm. Duplicate or triplicate samples were analyzed.

### Chromatographic Analyzes

### Gas Chromatography (GC)

Qualitative and quantitative analyzes of samples from the earlier stages of the formulation procedure, and with respect to triethanolamine and derivatives, were carried out using an Agilent Technologies 6890 Series GC System with a Flame Ionisation Detector (FID). The GC-FID was equipped with an Rxi-35sil MS column of dimensions 30 m*0.25 mm ID, film thickness 0.5 um (Restek). Method according to European pharmacopoeia 11.0, monograph 01/2017:1577 ‘Trolamine’. The mobile phase was, according to the method, pH 8 phosphate buffer:Acetonitrile=1:1 (v/v %).

For quantitation, single point calibration was used with heptane as internal standard.

Qualitative analyzes were also performed using a 6890N GC System connected to a 5973N mass selective detector (Agilent technologies), electron impact (EI) at 70 eV for mass ionization. The column used was an Rxi-5Sil MS (Restek) with the dimensions 30 m*0.25 mm ID, 0.25 um DF.

For identification purposes spectral mass data obtained were compared to reference data from the Wiley/NIST spectral library (SBN: 978-1-119-75033-8).

### Liquid Chromatography (LC)

Qualitative LC-analyzes were performed on samples from the stage in the formulation procedure after ropivacaine was added. LC analyzes with ultraviolet light (UV) detection were performed on a Thermo Fischer Scientific Vanquish UPLC instrument employing a Waters BEH C18 1.7 um 2.1*150 mm chromatographic column at 0.3 mL/min flow rate and otherwise method according to European pharmacopoeia 11.1, monograph 01/2017:2335 ‘Ropivacaine Hydrochloride Monohydrate’.

LC analyzes with UV and MS detection were carried out on a Waters Acquity UPLC Quattro Micro MS-instrument with electrospray ionisation (ESI) in positive-negative switching mode. The method used was based on monograph 01/2017:2335 as above, however, the mobile phase was changed to a MS-compatible one; instead of phosphate buffer 0.1% NH3/MeCN with gradient elution was used.

### Impurity Analysis: 2,6-dimethylaniline

One of the impurities of ropivacaine specified in the pharmacopeial monograph Eu Pharm 11.1, 01/2017:2335 as Impurity H, the carcinogenic 2,6-dimethylaniline, was analyzed in prepared formulations by LC-UV and LC-UV-MS against 2,6-dimethylaniline reference material.

### Results

### Samples Prepared

TABLE 10
Samples of example 4

| Sample | Description | #/ID |
|---|---|---|
| Preparation 1 before | One preparation of water and TEA, | 1. 23E0492-01 |
| microwave treatment | volume ratio 1:2 | |
| Microwave treatment | Aliquot from 23E0492-1 | 2. 23E0492-02 |
| preparation 1 | | |
| Preparation 2 before | One preparation of water and TEA, | 3. 23E0492-0525:2 |
| microwave treatment | volume ration 1:2 | |
| Microwave treatment | Aliquot from 23E0492-0525:2 | 4. 23E0492-0525:1 |
| preparation 2 | | |
| Complete | Based on 23E0492-2. To this | 5. 23E0492-03 |
| formulation except | already microwave treated sample | |
| PEG4000 | ropivacaine (66.7 mg/mL) was | |
| | added. This mixture is then diluted | |
| | with water (MQ) and pH adjusted | |
| | to pH 5. Final concentration of | |
| | ropivacaine 5 mg/mL. | |
| Autoclaved complete | Complete formulation including | 6. 23E0990 - 2 |
| formulation with | ropivacaine (Approx 4.9 mg/mL) | |
| PEG4000 | and PEG 4000. | |
| Autoclaved TEA + water | | 7. 23E0990-1 |
| 2:1 volume ratio | | |

Samples number 2, 4 were treated in microwave oven, according to the heat treatment of triethanolamine described above.

In sample number 5, 23E0492-03, ropivacaine was added after microwave treatment of sample 2.

All samples were clear liquids at room temperature (20-22 degrees C.).

### Fluorescence Measurements

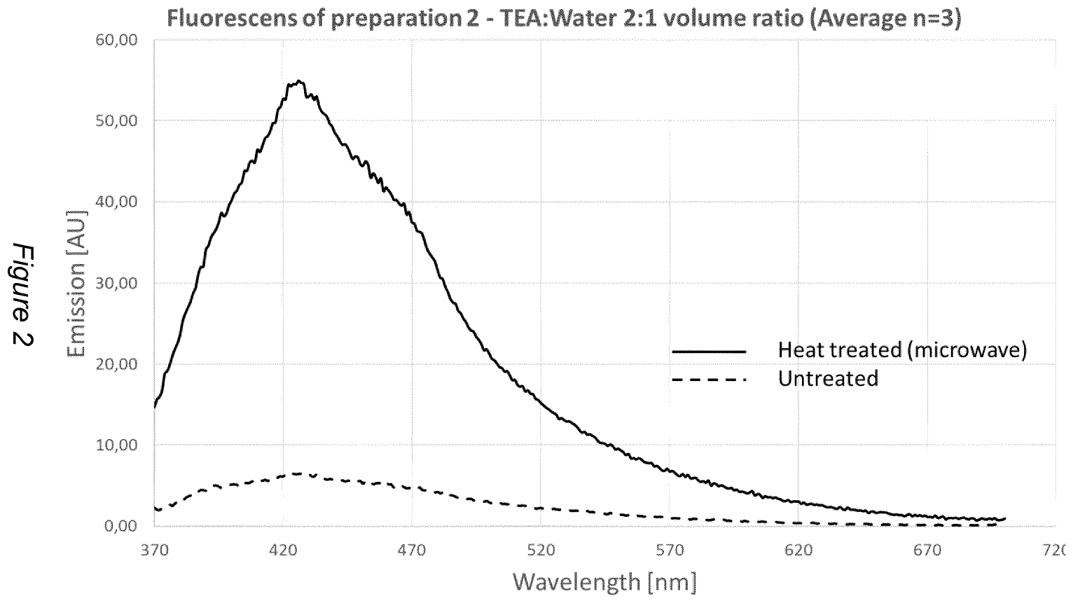

FIG. 2 shows a fluorescence diagram of a composition containing triethanolamine and water. The figure is showing fluorescence measurements (triplicates) of preparation 2 before and after microwave treatment (table 10, sample 4). Circles show fluorescence after microwave treatment and the small triangles show fluorescence before microwave treatment (table 10, sample 3). As can be seen in the figure, fluorescence is present in the preparation which has been microwave treated.

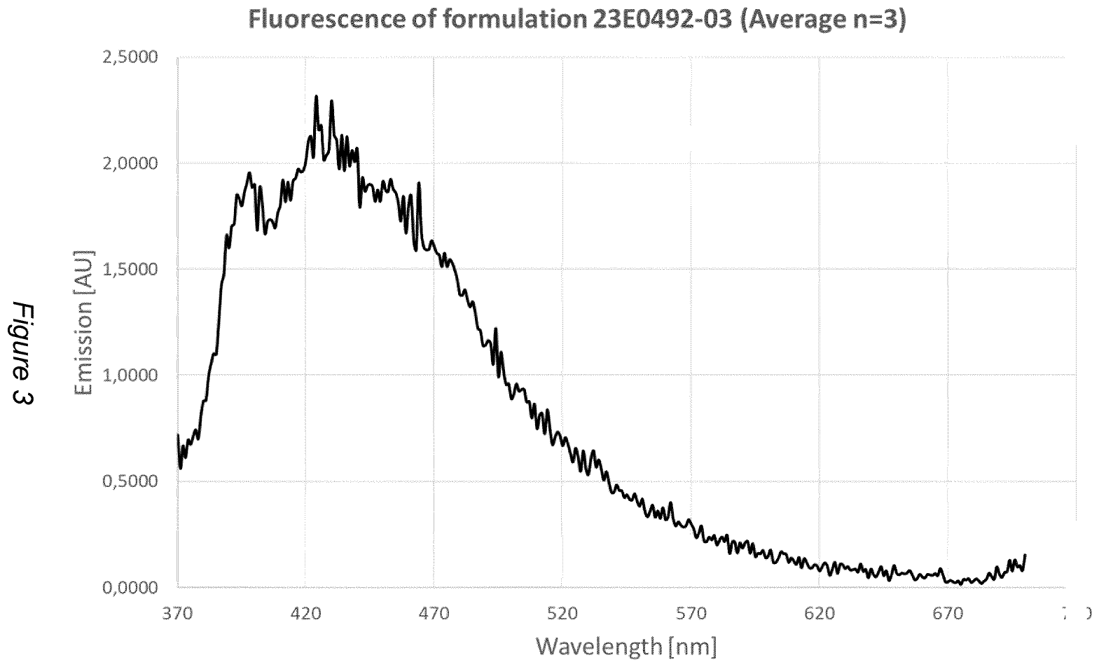

FIG. 3 shows a fluorescence diagram of a composition containing triethanolamine and water, which was microwave treated before the subsequent addition of ropivacaine. The figure is showing fluorescence measurements (triplicates) of sample 5 of table 10; i.e. formulation 23E0492-03. As can be seen in the figure, fluorescence is present in the preparation which has been microwave treated before the addition of ropivacaine.

### Chromatographic Analyzes

### Qualitative GC-Analyzes

In the analysis of a sample of the microwaved triethanolamine+water formulation (table 11, 23E0492-02, sample number 2), six peaks were detected at >0.05 area-% by GC-FID, apart from triethanolamine. See table 11. One of the impurities was identified by GC-MS (by reference to the Wiley/NIST spectral reference library) as diethanolamine (peak no 3 in table 11), which is one of three specified impurities of triethanolamine in the Eu pharmacopoeial monograph (Impurity B.) Diethanolamine was also present in the triethanolamine reference sample, but at the lower level of 0.01 area-%. Peak no 1 represents possibly monoethanolamine, Eu pharmacopeial monograph 01/2017:1577 specified Impurity A. It was also detected in the triethanolamine reference sample, at the level of 0.08 area-%. The remaining four peaks (peak no. 2, 5, 6, 7) could not be identified.

TABLE 11
Peaks detected by GC-FID analysis of sample 23E0492-02
(microwaved Triethanolamine + water formulation).

| Peak No. | ID | Ret. Time (min) | Rel. Area % (FID) |
|---|---|---|---|
| 1 | (Possibly | 2.1 | 1.0 |
| | Monoethanolamine) | | |
| 2 | n.a. | 6.2 | 0.05 |
| 3 | Diethanolamine | 6.7 | 0.7 |
| 4 | Triethanolamine | 7.1 | 96.7 |
| 5 | n.a. | 8.3 | 0.4 |
| 6 | n.a. | 8.3 | 0.4 |
| 7 | n.a. | 12.1 | 0.7 |

### 4.4.2. LC-Analyzes

In the analyzes of samples of the microwaved triethanolamine+water with added ropivacaine formulation (23E0492-03), only one peak was detected at >0.05 area-% level by LC-UV, apart from the triethanolamine and ropivacaine peaks. This was peak no 2 in table 12, at 0.2 area-% (UV 240 nm). The impurity could not be identified by LC-MS. It is probably related to ropivacaine since it is also present in the ropivacaine reference sample (at 0.5 area-%).

TABLE 12
Peaks detected by LC-UV analysis of sample 23E0492-03
(#5; microwaved Triethanolamine + water / Ropivacaine added).

| | | | Rel. Area % |
|---|---|---|---|
| Peak No. | ID | Ret. Time (min) | (UV, 240 nm) |
| 1 | Triethanolamine | 1.02 | 14.7 |
| 2 | n/a | 1.75 | 0.2 |
| 3 | Ropivacaine | 6.18 | 85.1 |

### 4.4.3. Quantitative GC Assay of Triethanolamine

GC-FID analysis was done using adapted Pharmacopeial method (01/2017:1577, Ph. Eur. 11.0) and single point calibration with internal standard (heptane). The method adapted in that the method was slightly modified, e.g. injection volume was changed from 2 to 1 μL, GC column was changed by 5% phenyl to 35% phenyl, and concentration of injected std triethanolamine and samples decreased a tenfold from 100 mg/mL to 10 mg/mL due to analyte overload with the original concentration. The analysis showed no significant change in triethanolamine content before and after heating treatments of the formulation procedure, i.e. microwave heating or autoclaving (see table 13).

TABLE 13
Results from quantification of triethanolamine
in selected samples by GC-FID analysis.

| | | Triethanolamine % | |
|---|---|---|---|
| Sample ID | Description | (w/w) | Results % |
| 23E0492-02 | Triethanolamine in | 70.1 | 73.4 |
| | water, microwave | | |
| | heated (140° C., 20 | | |
| | min) | | |
| 23E0990-1 | Triethanolamine in | 70.7 | 73.7 |
| | water, autoclaved | | |
| | (121° C., 15 min) | | |
| 23E0492 0525-2 | Triethanolamine in | 70.1 | 71.5 |
| | water before | | |
| | microwave heating | | |
| 23E0492 0525-1 | Triethanolamine in | 70.1 | 74.9 |
| | water after | | |
| | microwave heating | | |
| | (140° C., 20 min) | | |

### 4.5. Impurity analysis: 2,6-dimethylaniine

Specified Impurity H of ropivacaine of Ph. Eur. 11.1, 01/2017:2335-2,6-dimethylaniline—was not detected (<0.05 area-%), neither in the ropivacaine reference standard nor in the formulated samples of ropivacaine of microwave heated triethanolamine, as analyzed by UPLC-UV (240 nm) and UPLC-UV-MS (Full scan m/z 50-800). As such, it was concluded that 2,6-dimethylaniline is not produced in the treatment of a formulation of the present disclosure.

TABLE 14
UPLC-UV analyzes for 2,6-dimethylaniline
impurity in selected samples

| # | Name | Description | Impurity H |
|---|---|---|---|
| 1 | Standard | Ropivacaine reference | Not detected |
| | | standard, freshly prepared | |
| 2 | 23E0492-03 | Formulation with | Not detected |
| | | Ropivacaine added after | |
| | | microwave treatment | |
| | | (140° C., 20 min) pH 4-5 | |
| 3 | 23E0990-1 | Triethanolamine in water, | Not detected |
| | | autoclaved (121° C., 15 min) | |
| 4 | 23E0990-2 | Ropivacaine in | Not detected |
| | | triethanolamine, PEG4000 | |
| | | and water pH 5.2, | |
| | | autoclaved (121° C., 15 min) | |

TABLE 15
UPLC-UV-MS analyzes for 2,6-dimethylaniline
impurity in selected samples

| # | Name | Description | Impurity H |
|---|---|---|---|
| 1 | Standard | Ropivacaine reference standard, | Not detected |
| | | freshly prepared | |
| 2 | 23E0492-03 | Formulation with Ropivacaine | Not detected |
| | | added after microwave treatment | |
| | | (140° C., 20 min) pH 4-5 | |

### Example 5: In Vivo Test in Human

A formulation according to the present disclosure, such as a formulation according to example 2, composition 1 or 2, is formulated as a gel. The composition comprises Ropivacaine and lidocaine. The concentration of Ropivacaine in the formulation is 25 mg/ml. The concentration of lidocaine in the formulation is 25 mg/ml.

Emla® (25 mg/ml lidocaine and 25 mg/ml prilocaine formulated as a gel) is used as a positive control. The dosing of the formulation according to the present disclosure is equivalent to the dosing of Emla®, i.e., the dosing of prilocaine in Emla® and the dosing of Ropivacaine in the formulation of the present disclosure are equivalent.

The formulation is tested on 25-40 healthy volunteering medical students after appropriate ethical approval. The formulation is tested by application of the formulation to the dorsal (upper) side of the hand, to the skin between the base of finger 2 and 3 on the test subject (close to MCP joints-Meta carpo phalangeal joints).

At several time points, such as after 10 minutes, 20 minutes and after 60 minutes, numbness of the skin and entire fingers is tested by prick test on the finger tips.

### Expected Results

It is expected that numbness in all the fingers is achieved within some time, such as within 10 minutes or within 20 minutes, from application of a composition of the invention to the skin. It is expected that numbness of the entire fingers is not achieved within some time from application of Emla® to the skin.

The compound of the formulation will provide numbness of all the fingers of the hand to which to composition is administrated, i.e., the subject may not feel anything in said fingers or at least have reduced sensation. Said subject may be partly or wholly able to stretch and bend the finger during the time that the formulation of the invention is in effect.

It is expected that the results show that the formulation at least partly penetrate the skin of a human subject.

Numbness of the fingertips is an evidence for penetration of the composition through the skin, since the composition is applied to the skin between finger 2 and 3 (approximately 5 cm away) but provides numbness of the finger tips, i.e. the effect is provided at another position at a distance from the application site.

It is expected that the results show that the formulation according to the present disclosure is more effective, i.e., provide numbness faster and for a longer duration of time, compared to standard treatment (Emla®).

### Example 6: In Vivo Test in Human; Knee Operation

The purpose of this experiment is to test a composition of the invention as pain relief used in knee surgery.

A formulation according to the present disclosure, such as a formulation according to example 2, composition 1 or 2, is formulated for injection. The concentration of Ropivacaine is 5 mg/ml. The volume to be injected is proportional to the body weight of the subject, such that each patient receives 30-130 ml of formulation depending on said body weight. Commercially available Ropivacaine formulated for injection (5 mg/ml) is used as a positive control. The dosing of the formulation according to the present disclosure is equivalent to the dosing of the positive control. The compositions tested have approximately the same volume for injection and have the same pH.

The formulations are injected as sciatic/femoral nerve block, which is standard for this kind of procedure, as apparent to a person of skill in the art.

The subjects are otherwise healthy, middle aged male and female with osteoarthritis (OA) of the knee, subject to knee surgery. Before said surgery, the formulation is injected into the subject under sterile conditions and ultrasound guidance.

The operation is started 15-20 minutes after injection (when the anesthetic effect is achieved). The operation time is 60 to 90 minutes.

The study is done according to standard protocol and with appropriate ethical approval.

Ideally, an anesthetic effect should last for a long time, without interference with mobility of the patient. The following outcome measures are monitored in the study at time 0-72 hours post injection:

### Duration of Pain Relief

Time to rebound effect (i.e, when the anesthetic effect has passed)

The effect that the composition has on motor function of the patient (i.e. the interference with mobility of the patient)

Time before need of rescue medication (i.e. the time before the patient will need additional pain relief, such as opiates)

### Expected Results

It is expected that there is an initial partial paralysis of the leg, and total sensory loss of the leg seen for every composition tested in the experiment; both for a composition of the invention and for a composition according to prior art. This is due to the large volumes injected and well known effect of the anesthetic drug.

It is expected that total sensory loss of the leg is achieved for 4-5 hours from application of the prior art composition.

It is expected that total sensory loss of the leg is achieved for more than 4-5 hours, such as for 10-12 hours from application of a composition of the invention.

It is expected that the results show that the formulation according to the present disclosure is more effective, i.e., provide for sensory loss of the leg for a longer duration of time, compared to standard treatment. It is expected that the formulation according to the present disclosure provides sensory loss for more than 6 hours, such as for at for at least 10 hours.

1. A pharmaceutical composition comprising:
at least one anesthetic agent selected from the group consisting of ropivacaine, bupivacaine, etidocaine, levobupivacaine, lidocaine, lignocaine, mepivacaine, articaine, dibucaine, levobupivacaine, prilocaine, benzocaine, chloroprocaine, cocaine, procaine, proparacaine, tetracaine and any pharmaceutically acceptable salt, hydrate, solvate or prodrug thereof,
at least one alkanolamine selected from the group consisting of triethanolamine, tripropanolamine and trimethanolamine;
water; and optionally
a pharmaceutically acceptable diluent, carrier and/or excipient,
wherein said composition comprises carbon quantum dots.

2. The pharmaceutical composition according to claim 1, wherein said at least one anesthetic agent is selected from the list consisting of ropivacaine, bupivacaine, etidocaine, levobupivacaine, lidocaine, lignocaine, mepivacaine, articaine, dibucaine, levobupivacaine, prilocaine and any pharmaceutically acceptable salt, hydrate, solvate or prodrug thereof, or from the list consisting of ropivacaine, bupivacaine, etidocaine, levobupivacaine, lidocaine, lignocaine, mepivacaine, and any pharmaceutically acceptable salt, hydrate, solvate or prodrug thereof.

3. (canceled)

4. The pharmaceutical composition according to claim 1, further comprising polyethylene glycol (PEG).

5. (canceled)

6. The pharmaceutical composition according to claim 1, wherein said carbon quantum dots comprise said at least one alkanolamine.

7. The pharmaceutical composition according to claim 1, wherein said carbon quantum dots comprises said at least one anesthetic agents.

8-9. (canceled)

10. The pharmaceutical composition according to claim 1, wherein said composition is formulated to be administrated by injection, parenteral injection or subcutaneous injection, or by topical administration, a patch, a cream, by a gel or a spray.

11-13. (canceled)

14. The pharmaceutical composition according to claim 6, wherein the dose of said anesthetic agent is a single dose of from 5 to 600 mg.

15. The pharmaceutical composition according to claim 6, wherein the concentration of said anesthetic agent is at a dose of 2-15 mg/ml.

16. The pharmaceutical composition according to claim 1, wherein said anesthetic agent is selected from the group consisting of ropivacaine, bupivacaine and lidocaine.

17-18. (canceled)

19. The pharmaceutical composition according claim 1, wherein said anesthetic agent is ropivacaine.

20-25. (canceled)

26. The pharmaceutical composition according to claim 1, wherein said composition consists of
ropivacaine;
triethanolamine;
water;
PEG-4000;
carbon quantum dots;
and optionally
a pharmaceutically acceptable diluent, carrier and/or excipient.

27. The pharmaceutical composition according to claim 26, wherein a molar ratio of triethanolamine to ropivacaine in said composition is at least 2.1.

28. The pharmaceutical composition according to claim 1, wherein said composition remains stable upon storage for at least 1 day.

29-39. (canceled)

40. A method of treating, alleviating or preventing pain, comprising administrating a therapeutically effective amount of a composition according claim 1 to a patient in need thereof.

41. The method according to claim 40, wherein said pain is caused by surgery, an injury or trauma.

42. A method for production of a composition comprising carbon quantum dots and at least one anesthetic agent selected from the group consisting of ropivacaine, bupivacaine, etidocaine, levobupivacaine, lidocaine, lignocaine, mepivacaine, and any pharmaceutically acceptable salt, hydrate, solvate or prodrug thereof; the method comprising:
a) bringing an alkanolamine selected from the group consisting of triethanolamine, tripropanolamine and trimethanolamine into contact with water, thereby obtaining a mixture;
b) heating said mixture, thereby obtaining carbon quantum dots;
c) adding at least one anesthetic agent selected from the list consisting of ropivacaine, bupivacaine, etidocaine, levobupivacaine, lidocaine, lignocaine, mepivacaine, and any pharmaceutically acceptable salt, hydrate, solvate or prodrug thereof to said mixture;
wherein step b) and c) can be performed in any order;
d) optionally adding PEG to said mixture;
thereby obtaining a composition comprising carbon quantum dots and said anesthetic agent.

43-44. (canceled)

45. The method according to claim 42, wherein said heating is at a temperature of between 100° C. and 200° C.

46. The method according to claim 42, wherein said heating is by means of a microwave.

47. The method according to claim 16, further comprising a step of centrifuging said composition comprising carbon quantum dots and said anesthetic agent.

48-50. (canceled)

51. A carbon quantum dot formed from a composition comprising:
at least one anesthetic agent selected from the group consisting of ropivacaine, bupivacaine, etidocaine, levobupivacaine, lidocaine, lignocaine, mepivacaine, articaine, dibucaine, levobupivacaine, prilocaine, benzocaine, chloroprocaine, cocaine, procaine, proparacaine, tetracaine and any pharmaceutically acceptable salt, hydrate, solvate or prodrug thereof,
at least one alkanolamine selected from the group consisting of triethanolamine, tripropanolamine and trimethanolamine; and
water.

52-61. (canceled)
